(19) United States
(12) Patent Application Publication
(10) Pub. No.: US 2006/0019295 A1
(43) Pub. Date: Jan. 26, 2006

(54) Genomic barcoding for organism identification

(76) Inventor: Gernot Guenther Presting, Honolulu, HI (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/182,283

(22) Filed: Jul. 14, 2005

Related U.S. Application Data
(60) Provisional application No. 60/588,431, filed on Jul. 14, 2004.

(51) Int. Cl. C12Q 1/68; C12M 1/34

(52) U.S. Cl. 435/6; 435/287.2

(57) ABSTRACT

The invention disclosed herein relates to the comparison of whole genomes to identify short oligonucleotide sequences that are specific to a single organism. In some embodiments of the invention, combinations of species-specific oligonucleotides are used to produce specific amplification products. In some embodiments, isolate-specific oligonucleotides are used to detect and identify target organisms.

### CROSS-REFERENCE TO RELATED APPLICATIONS

This application claims the benefit of priority under 35 U.S.C. § 119(e) to U.S. Provisional Application Ser. No. 60/588,431, entitled GENOMIC BARCODING FOR SPECIES IDENTIFICATION, filed Jul. 14, 2004 which is hereby expressly incorporated by reference in its entirety.

### BACKGROUND OF THE INVENTION

1. Field of the Invention

The invention disclosed herein relates to the comparison of whole genomes to identify short oligonucleotide sequences that are specific to a single organism. In some embodiments of the invention, combinations of species-specific oligonucleotides are used to produce specific amplification products. In some embodiments, isolate-specific oligonucleotides are used to detect and identify target organisms.

2. Description of the Related Art

Traditionally, bacteria have been identified based on morphology and biochemical properties, ranging from Gram stain to their ability to metabolize certain chemicals (Brock T D, Smith D W, Madigan M T. 1984. Biology of Microorganisms 4^{th} edition. Prentice-Hall, Inc., Englewood Cliffs, N.J.). These tests are able to classify bacteria only into broad categories and most require purification of the bacterium. In the 1970s, antibodies began to be developed and used for pathogen detection, and several identification kits based on antibodies have been commercialized. The resolution of antibody-based identification methods is limited, however, as most antibodies identify species-specific epitopes and are unable to differentiate between sub-specific taxa, such as, for example, races or biovars. Bioassays, including host range determination, are often used to determine subspecies or races of bacterial pathogens. All of these assays suffer from a limitation of specificity and speed. More recently, the availability of DNA sequence for many economically important organisms and the advent of PCR, which enables amplification of minute amounts of DNA, has made DNA-based identification assays an attractive alternative for pathogen detection and identification.

One of the first applications of DNA-based identification methods has been to sequence a region of the ribosomal RNA genes. Prokaryotes and eukaryotes need ribosomal RNA genes for translation, thus this represents a universal marker that can be used in comparative sequence analysis to estimate evolutionary relationships among members of each kingdom. Although this feature makes rDNA a good universal marker in broad comparisons, it often fails to differentiate between closely related isolates. For example, rDNA may be used to identify an unknown as a member of a bacterial genus, but often will not be useful for species identification, let alone for identification of sub-specific taxa. Other genes or genomic regions, which evolve at different rates (e.g., avirulence genes, transposable elements) can be used to obtain better resolution. However, in most of these cases a single sequence is used for comparison, and it is not usually clear that the chosen sequence has any association with the phenotype on which the nomenclature is based (e.g., race). Although many pathogenic organisms have been completely sequenced, most PCR tests currently only assay the presence of one short sequence (usually less than 500 bp) representing around 0.01% of a bacterial genome. Furthermore, in most cases the region assayed has no causal relationship with the features that make an organism a potential biohazard (e.g., 16S rDNA with insect transmissibility). A further danger of using a single gene assay for identification is that a single mutation in the primer binding site can result in a negative test result even though the pathogen remains virulent.

The current classification system of the species complex Ralstonia solanacearum illustrates the problem with current identification methods. Originally, the Ralstonia species complex was divided into five races based on host range. These races were further classified into biovars based on their ability to oxidize hexose alcohols and three disaccharides. With the advent of DNA sequences a more refined method of classifying Rs isolates became possible. The ITS region of the ribosomal DNA allows differentiation of four phylotypes (I-IV). Even higher resolution was obtained using endoglucanase gene sequence, which to date has allowed identification of over 20 sequence variants (or sequevars) among the >140 isolates tested (Fegan and Prior, 2004). Additional studies using the hrp genes to identify sequevars are ongoing. However, these only represent the very beginnings of a thorough classification effort, as many of the traits important to disease (such as insect transmissibility and virulence) may be encoded by genes that are not linked to these three regions. The evolving nature of the Rs classification system, going from races to biovars, phylotypes and sequevars, has resulted in fairly inconsistent annotation of existing collections.

This point is vividly illustrated in the following example: R. solanacearum causes a serious wilt on many plants, including potato, tomato and tobacco. The most common race of R. solanacearum on tomato and tobacco is Race 1. Although Race 1 is ubiquitous in the southern growing regions of the US, the pathogenicity and virulence of this bacterium varies by location. For instance, R. solanacearum causes severe problems on tobacco in North and South Carolina, but the disease is rarely seen in Georgia and Florida (Fortnum B A and S B Martin. 1998. Disease management strategies for control of bacterial wilt of tobacco in the southeastern USA. Pages 394-402 in: Bacterial wilt disease: molecular and ecological aspects, P. Prior, C. Allen and J. Elphinstone, eds. Berlin Heidelberg: Springer-Verlag; Kelman A, Person L H. 1961. Strains of Pseudomonas solanacearum differing in pathogenicity to tobacco and peanut. Phytopathology 51:158-161). A recent study found a miniature transposable element to be, at least in part, responsible for the sharply divided demarcation between disease/no disease in these bordering states. This transposable element had inserted into the avirulence gene avrA in isolates recovered from nearly all infected fields in North and South Carolina. In contrast, this transposable element was only rarely seen in collections from Georgia and Florida. The authors of this study hypothesize that “disruption” of the avrA gene by the transposable element may have caused a shift in host recognition (Robertson A E, Fortnum B A, Wechter W P, Denny T P, Kluepfel D A. 2004. Relationship between the diversity of the avirulence gene, avrA, in Ralstonia solanacearum and bacterial wilt incidence in the southeastern United States. Mol Plant Microbe Interact. 17(12):1376-84). The rDNA or endoglucanase gene sequences would have no predictive value in this system. However, transposon insertions can be detected using rep-PCR.

Similar detection and identification problems are common to other kingdoms and phyla as well. For example, taxonomic identification of plant species is generally done using a defined set of anatomical features, often including flower characteristics. Experts on a particular taxon can identify even seedlings, though this is increasingly difficult the younger the seedling is. Furthermore, given the large number of entries on the Hawaii invasive species list, it is unlikely that there are more than a handful of experts who can identify all of them at all stages. Similarly, many seeds have morphological features that allow their classification, but again this becomes difficult where large batches of seeds need to be examined, particularly mixed seed.

Another example of tedious identification of species is evident in the classification of fish larvae, which are difficult to identify because their morphological characters change dramatically in the course of development.

Thus, there is a need for the development of much more reliable methods for rapid and specific detection and identification of organisms.

### SUMMARY OF THE INVENTION

The invention described herein relates to reliable methods for rapid and specific detection and identification of organisms. Thus, embodiments of the invention relate to methods for assembling such diagnostic tools, including for example, identifying and selecting oligonucleotide probes for use as genetic tags for selecting and differentiating an organism.

Some embodiments relate to methods for identifying oligonucleotide probes for selecting or differentiating an organism or for use as genetic tags in a bar coding assay. The methods can include the steps of selecting a nucleotide sequence in a genome of a first organism, wherein said nucleotide sequence is at least 20 nucleotides in length; analyzing a substantially whole genome of a second organism for the presence or absence of said nucleotide sequence; and classifying the at least one nucleotide sequence, wherein nucleotide sequences absent in the genome of the second organism are classified as taxon-specific probes and nucleotide sequences present in the genome of the second organism are classified as homologous probes.

The nucleotide sequence can be 12 to 60, or more, nucleotides in length, preferably 15 to 40 nucleotides in length, and more preferably 20 to 30 nucleotides in length. In some embodiments, the nucleotide sequences are at least 12, 13, 14, 15, 16, 17, 18, 19, 20, 21, 22, 23, 24, 25, 26, 27, 28, 29, or 30, 40, 50, 60, or more, nucleotides in length. In some embodiments the oligonucleotides are exactly 20, 21, 22, 23, 24, 25, 26, 27, 28, 29, or 30, 40, 50, 60 nucleotides in length. Preferably, the nucleotide sequences are 24 nucleotides in length.

In some embodiments, the selecting, analyzing, and classifying steps are repeated for at least 100, 200, 300, 400, 500, 600, or more sequences in the genome of the first organism. In some embodiments, the methods steps are repeated for all possible sequences in the genome of said first organism.

The methods can further include the step of reverse analyzing the genome of the first organism for sequences from the genome of the second organism. In some embodiments, the methods can further include analyzing a substantially whole genome of a third organism for the presence or absence of said nucleotide sequence.

In some embodiments, the analyzing step comprises computational analysis. In some embodiments, the analyzing step further comprises experimental analysis.

The first and second organisms can be genetically diverse members of the same species. Alternatively, the first and second organisms can belong to different species. The second organism can be selected based on greatest genetic diversity as compared to the first organism.

Other embodiments relate to methods for selecting a set of oligonucleotide probes for definitively identifying an organism or differentiating an organism from any other organism. The methods can include the step of analyzing at least two substantially whole genomes to identify at least one nucleotide sequence (probe) of at least 20 nucleotides, which sequence is present in a first genome and absent in a second genome.

Still other embodiments relate to arrays comprising a plurality of nucleic acid probes, wherein said plurality of nucleic acid probes are complementary to the oligonucleotides identified according the method described above, and wherein each sequence is attached to a surface of the array in a different localized area. The plurality of nucleic acid probes can include at least 100, 200, 300, 400, 500, 600, 700, 800, 900, 1000, 1500, 2000, 2500, 3000, 4000, 5000, 6000, 7000, 7500, 8000, 10,000, 20,000, 25,000, 50,000, 100, 000, 200, 000, 300, 000, 400,000 or more probes.

The plurality of probes can include oligonucleotides common to members of a particular sub-specific taxon but absent in closely related organisms. The plurality of probes comprises taxon-specific probes belonging to multiple genomic regions of a target organism. The multiple genomic regions can be evenly distributed throughout the genome of the target organism. For example, the multiple genomic regions are spaced at 10 kb intervals throughout the genome of the target organism.

The plurality of probes can include probes containing at least one, two, three, four, five, six, seven, eight, nine, ten, twelve, fifteen, or more mismatches or nucleotide differences as compared to the most closely related sequence of the genome of a non-target organism.

In some embodiments, the probes are selected based on G+C content. In some embodiments, the probes selected based on absence of secondary structure.

Yet other embodiments of the invention described herein relate to methods definitively identifying at least one organism from any other organism. The methods can include the steps of isolating and amplifying DNA from at least one organism in a sample; hybridizing the DNA with a set of oligonucleotide probes identified according to the method of claim 1; and analyzing the hybridization results to determine the identity of the organism. In some embodiments, the hybridizing step is a single step. The methods can use probes and arrays as described above. The DNA can be labeled prior to the hybridization step.

The methods can further include hybridizing the DNA with the homologous probes identified according the method described above to selectively amplify taxon-specific DNA in a sample. In some embodiments, selective amplification results in increased assay sensitivity.

Still other embodiments relate to methods for creating a genetic bar code for reliably assessing and/or identifying at least one organism. The methods can include the steps of selecting a nucleotide sequence in a genome of a first organism, wherein said nucleotide sequence is at least 20 nucleotides in length; analyzing a substantially whole genome of a second organism for the presence or absence of said nucleotide sequence; classifying the at least one oligonucleotide sequence, wherein oligonucleotide sequences absent in the genome of the second, genetically diverse organism are classified as taxon-specific probes and sequences present in the genome of the second, genetically diverse organism are classified as homologous probes; and selecting a combination of taxon-specific and homologous probes, wherein said combination allows for genetic distinction of an organism from any other organism.

In some embodiments of the invention described herein, the methods and arrays can be used to distinguish closely related organisms, such as members of a sub-specific taxon or isolates of a species. In some embodiments, the methods and arrays described above can be used to identify organisms across kingdoms.

### BRIEF DESCRIPTION OF THE DRAWINGS

FIG. 1 is a plot of all data points for all specific probes for each of Agrobacterium, Bradyhizobium, and Pseudomonas.

FIG. 2 is a phylogenic dendrogram of the Rs species complex based on endoglucanase (egl) sequences from 130 different strains.

FIG. 3 is a screen shot of a web-based prototype “bar-coding machine.”

### DETAILED DESCRIPTION OF THE PREFERRED EMBODIMENT

Embodiments of the invention described herein relate to the identification of unique combinations of primers or probes that can be multiplexed in such as way to clearly identify any organism from any sample from which DNA can be obtained to the desired level of resolution (e.g., genus, species, variety, race, pathovar) in a single test using known DNA technologies. Methods for identifying race-, biovar-, phylotype-, sequevar-, and subsequevar-specific primers or probes are also disclosed herein.

The methods described herein utilize whole or substantially whole genomes in the development of general assays that can differentiate among all form of life at all levels in a single step. “Substantially whole genomes” as used herein refers to sequences that are less than 100% of the organism genome. For example, substantially whole genomes can be 50%, 60%, 70%, 75%, 80%, 90%, 95%, 97% 98%, or 99% of the entire genome. The assays can be performed with any of a number of high-throughput genomics technologies that are standard practice and well known to those of skill in the art. The assays are based on the identification of a unique set of sequence tags for multiple genomic regions of an organism that is to be detected. Thus, nucleotide sequences located throughout a genome, rather than those located within a single region of the genome, are used to rapidly and reliably detect and identify specific organisms. Unique probes for various different organisms, for example unique probes for all plant pathogens, can be combined for use in a single high-throughput assay. The combination of these tags can then be used to positively identify the DNA of any organism from any other organism, at any level of resolution, in one simple assay.

Accordingly, computational methods for identifying potentially diagnostic regions of sequenced, substantially whole genomes and methods for using potentially diagnostic sequence tags with existing high-throughput genomics technology are described herein. Standard bioinformatics tools and procedures can be used to compare substantially whole genomes to identify short nucleotide sequences (oligonucleotides) over the entire stretch of the genome that are specific to a single organism as well as to identify those that are common to closely-related organisms. By identifying these regions computationally, the efficiency and speed of selecting sequences that can be utilized in diagnostic tests is greatly improved.

Feasibility of the Approach

The number of polymorphic nucleotides required to differentiate among a set number of species is quite small. Table 1 shows the number of organisms that can be resolved with a given number of nucleotides, assuming four possible nucleotides for each position in the sequence. Thus, theoretically one polymorphic nucleotide can differentiate among as many as 4 different organisms, while ten variable nucleotides suffice to differentiate among as many as 1 million variants.

TABLE 1
Number of different accessions that can theoretically be resolved by the
indicated number of polymorphisms in the sequence

| Number | Theoretical |
|---|---|
| polymorphic | resolvable |
| nucleotides | complexity |
| 1 | 4 |
| 2 | 16 |
| 3 | 64 |
| 4 | 256 |
| 5 | 1024 |
| 6 | 4096 |
| 7 | 16384 |
| 8 | 65536 |
| 9 | 262144 |
| 10 | 1048576 |

Thus, in theory, any amplified DNA sequence that contains 10 polymorphic positions could be used as a unique sequence, or genomic barcode, to positively identify each of over a million different accessions. This, of course, is an oversimplified view. In fact, many polymorphic sites have fewer than four variants, and due to the evolutionary relationship of life on earth the variations at each polymorphic site are not equally distributed (i.e. 25% each of A, T, C and G). Nevertheless, this calculation illustrates how little sequence is required to differentiate among a very large number of species.

Selection of Diagnostic Sequence

Advances in sequencing technology have resulted in the rapid deciphering of whole genome sequences in recent years. Complete genomic sequences are now available for many pathogens, and more sequences are added to the public databases daily (as of 14 Jun. 2005, GenBank (hypertext transfer protocol: www.ncbi.nlm.gov/genomes/MICROBES/Complete.html) contained 214 and 22 completely sequenced eubacterial and archael genomes, respectively, plus 18 completed fungal genomes). A much larger number of on-going genome projects are listed at specialized websites and summarized, in part, at Genomes OnLine Database (GOLD at hypertext transfer protocol: genomesonline.org).

In embodiments described herein, a whole or substantially whole first genome is broken down into short nucleotide sequences (oligonucleotides) that are screened against whole or substantially whole genomes of other organisms, either closely related or not, to identify shared and unique oligonucleotide probes that can be used to identify a single organism or a group of organisms. The oligonucleotides can be selected without regard to reading frames or particular genes. The oligonucleotides can be 12 to 60, or more, nucleotides in length, preferable 15 to 40 nucleotides in length, and more preferably 20 to 30 nucleotides in length. Thus, in some embodiments, the oligonucleotides are at least 12, 13, 14, 15, 16, 17, 18, 19, 20, 21, 22, 23, 24, 25, 26, 27, 28, 29, or 30, 40, 50, 60, or more, nucleotides in length. In some embodiments the oligonucleotides are exactly 20, 21, 22, 23, 24, 25, 26, 27, 28, 29, or 30, 40, 50, 60 nucleotides in length.

In some embodiments, oligonucleotides are randomly selected from the first genome sequence. The oligonucleotides can be selected such that they are evenly distributed throughout the entire first genome. In other embodiments, all possible short nucleotide sequences between 20 and 30 nucleotides in a given genome are screened. For purposes of illustration, oligonucleotides that are 24 nucleotides (24-mers) in length will be used. Thus, for a circular genome of n base pairs, where all possible 24-mers are identified, n oligonucleotides are screened, i.e. n sequence comparisons against another genome are performed. For a liner genome of x base pairs, where all possible 24-mers are identified, x-24 oligonucleotides are screened, i.e. x-24 sequence comparisons are performed.

Each nucleotide sequence screened is classified according to whether it is present or absent in the genome against which it is screened. Sequences determined to be present in the screened against genome can be classified as homologous sequences with respect to the two genomes. Sequences determined to be absent in the screened against genome can be classified as taxon-specific. Sequences absent in a genome are determined based on the existence of at least one, two, three, four, five, six, seven, eight, nine, or ten or more mismatches detectable using currently available array technology. Thus, in some embodiments, a taxon-specific sequence can be a sequence that is a sinble base difference with a sequence contained in the screened against genome.

In some embodiments, a reverse comparison of the two genomes is also performed. That is, the initially screened against genome is broken down into short nucleotide sequences that are then screened against the genome of the first organism. In some embodiments, each nucleotide sequence screened against one genome is also screened against at least one other genome. Optionally, each nucleotide sequence can be screened against all available genomes.

The resulting output of the above screens is a library of various different probes, some of which are unique to an individual organism and some of which are common to at least one other organism. Some of the probes can be identified as common to a particular taxon. Others can be identified as regions of significant difference at all taxonomic levels. Homology requirements of common probes can be increased, and the sensitivity of the computational analysis increased to identify taxon-specific oligos. Alternatively, homology requirements and the sensitivity of the computational analysis can be decreased to increase the identification of universal or taxon-common probes. Such refinement of probe design specifications can be used to increase or decrease assay sensitivity.

Sequence comparisons can be performed using computationally intensive but exhaustive sequence comparison algorithms that are well known to those of skill in the art, for example, but not limited to, pattern matching and/or BLAST (Basic Local Alignment Search Tool). A single search conducted with Agrobacterium, Bradyrhizobium and Pseudomonas (see Example 3), in which only 1/24^{th of the DNA space was queried, required a little over} 2 hours on a single G5 processor. Thus, performing an exhaustive search of 160 bacterial genomes would increase the complexity and duration of the problem by 53×53×24 (assuming a 53-fold larger query set (160/3), 53-fold larger database, and 24-fold increase in sampling density), or by a factor of 67,416, requiring a total of 134,832 hours (5,618 days) of compute time. Thus, a compute cluster of 10 CPUs devoted exclusively to this computation could do the entire computational analysis in 562 days. However, by tripling the computational capacity the computational work can be accomplished in 62 days even without utilizing a pre-filter to enrich for informative sequence comparisons. These numbers are calculated based on the use of conventional computing power. Use of supercomputers and fairly multiplexed computers known in the art can significantly reduce the computing time.

Various pre-screening methods can be used to reduce the number of uninformative nucleotide comparisons. For example, pair-wise pre-screening of whole genomes prior to the screening of possible 24-mers can reveal large stretches of homology between closely related genomes, enabling the collapse of query sequences into groups. This method can be particularly effective in comparing closely related species or strains. For example, sequence identity of the 33 Ralstonia solanacearum gene sequences in GenBank ranges from 100% to 92% over 664 base pairs. These 664 bp comprising 640 different 24-mers, can be collapsed into a much smaller number of queries. Tools that can efficiently identify regions of 100% sequence identity in whole genome comparisons are well-known to those of skill in the art, including, for example, suffix-tree algorithms (Delcher A L, Kasif S, Fleischmann R D, Peterson J, White O, Salzberg S L. 1999. Alignment of whole genomes. Nucleic Acids Research 27(11):2369-2376; Delcher A L, Phillippy A, Carlton J, Salzberg S L. 2002. Fast algorithms for large-scale genome alignment and comparison. Nucleic Acids Research 30(11):2478-2483; Kurtz S, Phillippy A, Delcher A L, Smoot M, Shumway M, Antonescu C, Salzberg S L. 2004. Versatile and open software for comparing large genomes. Genome Biology 5:R12) and suffix arrays (Abouelhoda M I, Kurtz S, Ohlebusch E. 2002. The enhanced suffix array and its applications to genome analysis in: Proceedings of the 2nd Workshop on Algorithms in Bioinformatics, pages 449-463, LNCS 2452, Springer Verlag).

The results of each completed comparison can be stored in a common data table, eliminating the need to repeat each search in the future. Thus, once all completed genome sequences are processed, further analysis can be focused on the newly sequenced genomes as they are published. In an oversimplified representation of the results as a table, with rows representing 24-mers in completely sequenced genomes and columns representing the organisms in which they occur, each additional sequenced genome will add one column and a maximum of n new rows, with n representing the number of 24-mers in the new genome.

These screening methods can be used to generate “genomic barcodes,” or a unique sequence or set of sequences, which are used to definitively identify any organism. The principle of genomic barcoding is the simultaneous assaying of a unique set of sequences to allow definitive identification of an organism in a very rapid assay. In some embodiments hundreds of sequences are selected to be assayed simultaneously. In some embodiments, thousands of sequences are selected to be assayed simultaneously. In some embodiments, the unique set of sequences comprises those sequences identified as being specific to a particular isolate. In some embodiments, the unique set of sequences comprises sequences identified as common between all isolates but absent in closely related genomes. In some embodiments, the unique set of sequences comprises a combination of taxa-specific and shared sequences. In some embodiments, genomic barcodes assembled according to these methods can be used to identify oligonucleotides that can differentiate, for example, phylotypes, sequevars and clonal lines of plant pathogens.

Several factors can be considered in the selection of diagnostic DNA sequences. For example, depending on the sample to be assayed, a sequence can be selected based on its presence in all organisms to be examined, that it is well enough conserved to allow amplification, for example, via PCR, from the entire range of organisms, but that it contains enough polymorphic sites to allow differentiation among all of the species regardless of their evolutionary relatedness. For example, for differentiation between two distantly related organisms, examination of a highly conserved sequence to prevent revertants (i.e. mutation of T>C followed by reversion from C>T) from confusing the picture is preferred. On the other hand, a fast evolving sequence can be examined to distinguish between two very recently diverged species. Thus, selection of the sequence to be used for differentiation depends in part on the evolutionary relationship of the organisms to be classified and the intended purpose of the assay.

Probes specific to a particular genome can include those that contain 1, 2, 3, 4, 5, 6, 7, 8, 9, 10, 12 or more mismatches to the most closely related sequence of another genome.

For purposes of illustration, the devastating wilt-causing phytobacterium Ralstonia solanacearum (Rs) is used in the following example. Sequenced and aligned Ralstonia genomes can be scanned for conserved primer pairs that can be used to amplify a short genomic region every 10 kb, resulting in the generation of 600 primer sets (6 Mb genome/10 kb) that can be used to amplify conserved sequences from any Rs isolate. 600 regions from a total of 10 key Rs isolates have been amplified and sequenced in order to identify additional polymorphisms.

These polymorphisms can be used to identify isolate-specific oligonucleotide probes that can be used to exhaustively characterize any Rs isolate for 600 evenly spaced markers in a single microarray assay. The result is an “Rs” chip containing 400-600 oligos from up to 15 isolates. This will allow identification of any Rs subspecies as well as a hypothetical chimeric megaplasmid resulting from a recombination event between two different phylotypes in the field.

Furthermore, these primers can be used in long-distance amplification methods, such as, for example, PCR, to detect and simultaneously map genomic insertions and deletions. This test is similar to rep-PCR in scope (it assays most of the genome), but the resolution is significantly higher. With this system it is possible to detect transposon insertions like the one thought to be responsible for the severe tobacco wilt phenotype of the Carolina isolate mentioned above (Robertson A E, Fortnum B A, Wechter W P, Denny T P, Kluepfel D A. 2004 Relationship between the diversity of the avirulence gene, avrA, in Ralstonia solanacearum and bacterial wilt incidence in the southeastern United States. Mol Plant Microbe Interact. 17(12):1376-84).

The examples below illustrate the development of a list of 24-mers useful for detection and differentiation of one particular, very important plant pathogen, Ralstonia solanacearum, which is also a select agent. These examples are illustrative only and are not intended to limit the invention in any way. Those of sill in the art will recognize that this system can be expanded to include all other completely sequenced genomes. For example, the analysis can be expanded to all completely sequenced select agents, followed by all completely sequenced plant-associated bacteria (including Agrobacterium, Bacillus, Bradyrhizobium, Clostridium, Corynebacterium, Xylella and Xanthomonas strains). This analysis can be further expanded to include all sequenced bacterial, viral and fungal plant pathogens.

Detection and/or Identification of Organisms

Powerful new technologies such as ultra-high density photolithography microarrays that allow the simultaneous assaying of tens of thousands of markers and real time PCR, which provides amplification results in a fraction of the time required by standard PCR, are ready for immediate use in pathogen detection strategies.

In some embodiments of the invention, combinations of species-specific oligomers are used in PCR tests to produce amplification products of staggered sizes. PCR, both regular PCR followed by gel electrophoresis and real-time PCR, in which a primer-signal molecule combination can be used to detect amplification of specific products, are well established techniques that are useful with the methods described herein.

In some embodiments, amplification primers can be designed to be specific for each of a number of members belonging to a particular taxon to be assayed and to yield a specific product size for each. For example, an amplification primer pair set from a first phylotype can be designed to yield amplification products of between 100 and 200 bp, a primer pair set from a second phylotype can be designed to yield amplification products of between 200 and 300 bp, and so on. All primers can be combined in a single reaction containing DNA from one or multiple phylotypes. The resulting amplification products can be run on a gel to identify which phylotypes are present in a sample.

In another embodiment, amplification primers that amplify a product in all phylotypes can be designed along with a phylotype-specific signal molecule carrying a phylotype-specific molecular beacon for each primer pair thereby allowing multiple phylotypes to be resolved simultaneously in one real-time PCR run.

In still other embodiments, oligomers can be used in micro-array hybridizations with environmental DNA samples to detect and identify target microbes. With micro-array technology continuing to increase speed and robustness of the assay, micro-array based assays can be an attractive complement to amplification-based tests. The advantage of micro-arrays lies in the very large number of oligonucleotides that can be queried simultaneously (currently up to 400,000), which allows the design of a single chip that can test for hundreds of organisms simultaneously using hundreds of markers for each organism.

As will be recognized by those of skill in the art, hybridization and wash conditions for the micro-arrays can be optimized to improve sensitivity of the assays disclosed herein.

Methods for conducting hybridization assays are well known to those of skill in the art. Those of skill in the art will recognize that hybridization assay procedures and conditions will vary depending on the application and are selected in accordance with the general binding methods known in the art. Hybridizations are typically performed under stringent conditions that are well known to those of skill in the art. Hybridization and wash conditions are known in the art.

Arrays are well known to those of skill in the art to comprise a support with nucleic acid probes attached thereto. In embodiments of the invention described herein, the arrays comprise a plurality of different nucleic acid probes coupled to the surface of a substrate in different, known locations. Arrays are also known in the art as “micro-arrays” or “chips.”

In addition, portable cyclers now allow field-based testing (Schaad N W, Opgenorth D. Gaush P. 2002. Real-time polymerase chain reaction for one-hour on-site diagnosis of Pierce's disease of grape in early season asymptomatic vines. Phytopathology 92:721-728), so these tests could be immediately applied.

In some embodiments, oligomers determined computationally to be of the desired specificity can be applied to glass slides using arraying hardware and hybridized with DNA samples to determine the presence and composition of organisms contained within such samples.

Sample Preparation

Nucleic acid is a universal and essential component of any living organism, primarily in the form of DNA. The phenotype of an organism is ultimately encoded by its DNA sequence, for example, toxin synthesis depends on the presence of functional genes required to make the toxin and infection of a host by a pathogen requires the presence of the appropriate pathogenicity genes. In some organisms, RNA is the functional genetic material—in those cases RNA can be converted into DNA with the enzyme reverse transcriptase.

Nucleic acid can be amplified efficiently, by various means, such as, for example, through polymerase chain reaction (PCR), enabling detection of single molecules of either DNA or RNA. Where available, antibodies can be used to affinity-purify cells from a target organism from a complex environmental mixture prior to DNA amplification. The tools to work with nucleic acids (isolation, amplification, storage, detection) are well developed.

In some embodiments of the invention, environmental DNA can be isolated and amplified from samples, such as, but not limited to, soil, water, and plant material. Soil isolation kits are known in the art and available from a variety of vendors. However, inhibition of Taq polymerase by picogram quantities of humic acid can interfere or inhibit the PCR amplification process. Polyvinylpolypyrrolidone, particle flocculation, and centrifugation has proven exceptionally effective for removal of contaminating humic compounds from a wide variety of soil types.

Selective Amplification

For environmental samples, probes identified as homologous or common to related groups of organism can be used to amplify taxon-specific DNA in a sample. For example, taxon-specific PCR amplification can be performed as a filtering step to increase certain, desired DNA in a sample. This selective amplification of environmental DNA can be used to increase the sensitivity of the assay.

### EXAMPLES

### Example 1

da Silva et al. describes the differences and similarities between two plant pathogenic Xanthomonas species, X. campestris pv campestris (Xcc) and X. axonopodis pv citri (Xcc). da Silva et al. 2002. Comparison of the genomes of two Xanthomonas pathogens with differing host specificities. Nature 417(6887):459-63. The species were found to share 2,929 genes, but Xcc contained 646 genes (15.4%) not found in Xac and Xac contained 800 genes (18.5%) not found in Xcc. These subregions of each genome are ideal locations from which to derive oligomers that are specific to one or the other genome.

### Example 2

An unknown bacterium was co-sequenced with the rice genomic DNA during the TMRI rice shotgun sequencing effort (Goff et al. 2002. A draft sequence of the rice genome (Oryza Sativa L. ssp japonica). Science 296(5565):79-92). Analysis of the recA gene sequence showed this organism to be related to both Xylella and Xanthomonas. In order to determine the genus to which this putative rice endophyte belonged, its sequence was divided into open reading frames and the presence or absence of each of the 39,864 hypthetical ORFs in each of several bacterial species was determined.

TABLE 2
Presence/absence call for hypothetical ORFs from unknown bacterium in a
variety of related bacteria (Pseudomonas and Rhizoctonia data not shown).

| Number of ORFs | present in | and absent in |
|---|---|---|
| 2218 | Xanthomona campestris | Xylella fastidiosa |
| 60 | Xylella fastidiosa | Xanthomona campestris |
| 207 | Xanthomonas campestris | Xanthomonas citri |
| 163 | Xanthomonas citri | Xanthomonas campestris |

Table 2 illustrates how the presence/absence data can be used to determine the relatedness of unknowns to known organisms using DNA sequence. A comparison of the unknown bacterium with Xanthomonas campestris and Xylella fastidiosa revealed 2218 ORFs that occur in the former and not in the latter. In the reverse comparison, only 60 ORFs were found in Xylella fastidiosa and not found in Xanthomonas campestris. Thus, the unknown bacterium clearly is much more closely related to Xanthomonas than Xylella. A X. campestris versus X. citri comparison shows much smaller differences (207 versus 163). This analysis is similar to phylogenetic trees in that it determines genetic distance based on sequence data, however it differs from phylogenetic trees constructed from single proteins in that this analysis represents a whole-genome scan.

These principles can be applied to the analysis of any other kind of data such as presence/absence of amplification products, for example, in PCR tests, or +/−hybridization signal data from micro-arrays.

### Example 3

The entire completed genome sequences of Agrobacterium tumefaciens C58 (circular and linear chromosomes, AT and Ti plasmids), Pseudomonas putida KT2440, and Bradyrhizobium japonicum USDA 110 were screened for oligonucleotide probes specific to each genome according to the methods described herein.

A micro-array chip was manufactured containing a total of 6,448 probes designed from these completed genomes. Approximately 500 of these probes were specific to each of the genomes (i.e. contain 4 or more mismatches to the most closely related sequence of the other strain. The remainder of the probes contained 1, 2 or 3 mismatches to the most closely related sequence of the other strains. These mismatched probes were included in order derive rules on how dissimilar probes have to be in order to not hybridize with DNA from non-target organisms (false positive), and the effect of single and multiple mismatches on probe specificity. For example, a single mismatch at the end of the probe may not have a large effect on hybridization efficiency. The G+C contents of the designed probes varied from 0% to 96%, again to allow derivation of some general rules about which range of G+C contents is best suited for these hybridizations (Presting G G. 2003. Mapping multiple co-sequenced T-DNA integration sites within the Arabidopsis genome. Bioinformatics 19(5):579-86.).

Twelve chips were successfully tested purified labeled DNA from each of the bacteria, as well as labeled DNA isolated from non pasteurized field soil samples spiked with two concentrations of Agrobacterium and Bradyrhizobium. DNA isolated from pure cultures was used to determine the specificity and cross-reactivity of each designed probe, and spiked soil and plant samples were used to assess the sensitivity of the micro-array and the specificity of the probes in a background of “soil DNA” or “plant DNA.”

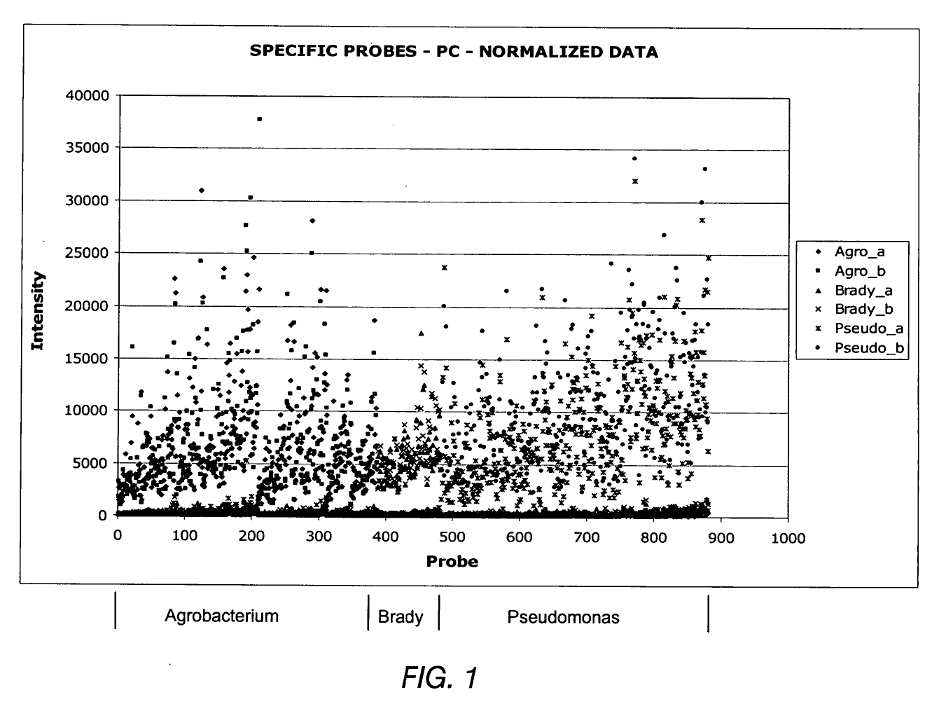

The above-described micro-array was able to differentiate between Agrobacterium, Bradyrhizobium, and Pseudomonas. (See FIG. 1.) All data points for all specific probes are plotted. Probes derived from one species (x axis) hybridized most strongly with DNA from itself. This was true for DNA samples isolated from pure cultures or spiked soil. A detection threshold in soil of between 10³ and 10⁶ cfu per gram of soil without any specific DNA amplification was determined.

The experiment confirmed between 100 and 400 “universal” and “genus-specific” probes for each genus. Results are summarized in Table 3.

TABLE 3
Probe Composition of Microarray

| | | |
|---|---|---|
| Agrobacterium | 24/24 (unique) | 574 |
| | rev. compl. Unique | 574 |
| | 23/24 (terminal) | 167 |
| | 23/24 (internal) | 1,711 |
| | 22/24 | 350 |
| | 21/24 | 349 |
| Bradyrhizobium | 24/24 (unique) | 446 |
| | rev. compl. unique | 446 |
| Pseudomonas | 24/24 (unique) | 697 |
| | rev. compl. unique | 697 |
| Universal | | 437 |
| Total | | 6,448 |

### Example 4

The Case for Sequencing Additional Ralstonia Isolates

Ralstonia solanacearum is one of the most important bacterial plant pathogenic threats to U.S. agriculture, yet it is genetically very poorly understood. For example, it is not known how host range is specified, or how easily host range is changed. The “species complex” has been classified into races, phylotypes and sequevars based on host range and comparative sequence analysis of two genetic loci, namely ribosomal DNA and the endoglucanase gene. The exact genetic relationship of the subdivisions to each other remains unclear. With so few genetic loci under analysis, this situation is unlikely to change. Yet decoding the genetics of Ralstonia will aid the understanding how this important pathogen evolves, how different geographic isolates differ from each other, and how different races interact in field communities.

Ralstonia solanacearum is characterized by a very wide host range that includes crops, weeds and native plants from more than 50 plant families. This bacterium is a pathogen on more than 200 plant species, including many of significant commercial importance, most notably potato. Rs affects monocots and dicots, herbaceous and woody plants from both tropical and temperate regions. It is a soil-borne gram-negative bacterium that can survive for years in soil and water. Rs invades the host xylem through the roots and causes severe wilt and death. Currently the best strategies for control of bacterial wilt include breeding for resistance and use of clean cuttings in vegetatively propagated crops such as bananas, ginger, ornamentals, plantains and potatoes. Plants can carry detectable populations of Ralstonia solanacearum without showing any symptoms, a phenomenon known as latent infection.

Rs has historically been classified into races based on host range. However, the host from which a Rs strain is isolated is not necessarily a good predictor of its race. For example, in 1996 Ralstonia was isolated from imported geranium cuttings. At that time there was no evidence that geranium isolates could also affect potato. It is equally conceivable that Ralstonia race 3 biovar 2 could be imported today on other crops unknown to serve as hosts for this pathogen. Hawaii is an entry and transfer point of shipping to the United States—propagative materials for the ornamental and fruit industries are routinely brought in from high elevation Central and South America, making on-site pathogen detection and quarantine critically important.

Due to its economic importance, wide host range and longevity, a number of large Rs culture collections are available. Many of the Rs strains have been characterized using AFLP, RFLP and rep-PCR to assess the population diversity (Alvarez et al. 2004. Integrated approaches for detection of plant pathogenic bacteria and diagnosis of bacterial diseases. Annu Rev Phytopathol. 42:339-66; Yu et al. 2003. Molecular diversity of Ralstonia solanacearum isolated from ginger in Hawaii. Phytopathology 93(9):1124-1130). The evolving nature of the Rs classification system in recent times, going from races to biovars, phylotypes and sequevars, has resulted in fairly inconsistent annotation of existing collections. Rs isolates classified according to the phylotype and sequevar nomenclature developed by Prior and Fegan are reclassified according to the methods described herein.

For unknown reasons blood disease has spread rapidly across the Indonesian archipelago during the past 17 years, after being confined to Sulawesi for most of the last century (Fegan M. (2004). Bacterial Wilt Diseases of Banana; Evolution and Ecology. In Bacterial Wilt: The Disease and the Ralstonia solanacearum species complex. Edited by C. Allen, P. Prior and C. Hayward, APS Press, St. Paul). This and many other important biological and biosecurity questions can be addressed once a complete set of genetic markers is available for this species complex.

Sequencing Status of Ralstonia Solanacearum

Complete sequence data for one Ralstonia solanacearum isolate (GMI1000—race 1, phylotype I), isolated from tomato, has been published. Sequence of the economically important potato isolate Race 3 biovar 2 has recently been completed and a closely related banana isolate Race 2 biovar 1 is currently being sequenced.

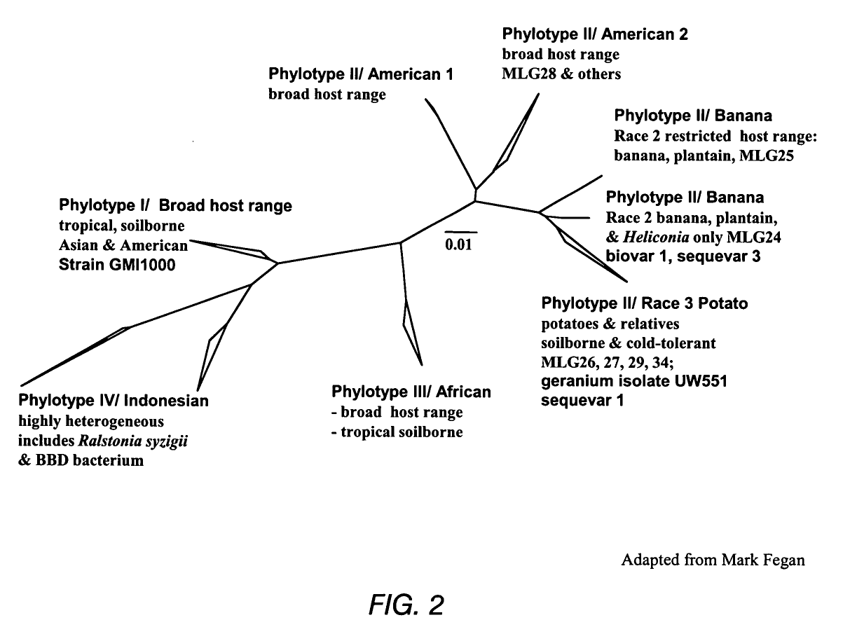

Since the objective is to collect as many distinguishing markers as possible, isolates representing the greatest genetic diversity within the Rs species complex are sequenced at 1.5× genome coverage. The phylogenetic relationship of these sequenced isolates (marked in red) to each other and other isolates is illustrated in FIG. 2. Briefly, one isolate from each of phylotypes II, III and IV is chosen. The phylotype II isolate chosen is from the American 1 broad host range branch (sequevar 7, MLG 1) that, based on the endoglucanase gene sequence, is the phylotype II isolate most distantly related to the other phylotype II isolates currently being sequenced (FIG. 2). Sequevar 7 contains the type strain and strain AW1. AW1 is the strain on which Tim Denny (Kang Y, Liu H, Genin S, Schell A M, Denny T P. 2002. Ralstonia solanacearum requires type 4 pili to adhere to multiple surfaces and for natural transformation and virulence. Mol. Microbiol. 46(2):427-437) has performed extensive analysis of host-pathogen interactions. Phylotype III is a fairly narrow group from Africa and it is currently unclear how much genetic diversity it harbors. JT528, CFBP3059 and J25 are candidates for phylotype III sequencing. Phylotype IV is a very heterogeneous group that contains Rs isolates as well as the closely related R. syzygii and the blood disease bacterium (BDB). R142 (sequevar 9), which is related to the BDB, is sequenced.

The sequencing of additional, carefully chosen and genetically diverse isolates yields hundreds of genetic markers throughout the genome, which are used for detection and identification of very specific isolates. These markers allow researchers to follow isolates across continents, test for exchange of genetic materials between different races and differentiate between select agents (Rs race 3 biovar 2) and harmless epiphytes.

The sequence of Rs strain GMI1000, a Race 1 strain isolated from tomato, was published in 2002 by Salanoubat et al. 2002. Genome sequence of the plant pathogen Ralstonia solanacearum. Nature 415(6871):497-502. This genome spans 5.81 megabases split into two replicons (3.7 and 2.1 megabases) of nearly identical G+C content. The larger replicon contains many essential genes, including those required for DNA replication and repair, cell division, transcription and translation plus all essential genes for purine and pyrimidine biosynthesis. It has been designated the ‘chromosome.’

The smaller genetic entity, thought to represent a megaplasmid, contains duplicate copies of essential genes that are also present on the ‘chromosome,’ but also encodes some enzymes controlling amino acid and cofactor biosynthesis that have no counterpart on the ‘chromosome.’ Loss of the megaplasmid would thus presumably make the bacterium auxotrophic for several metabolites.

The presumed megaplasmid carries numerous genes involved in overall fitness and adaptation to various environmental conditions. It carries all of the hrp genes that are required to colonize plants, and also encodes the constituents of the flagellum and most of the genes governing exopolysaccharide synthesis.

Notable in relation to marker development for pathogen identification is that the genome encodes a total of four complete ribosomal DNA loci, three of which are on the ‘chromosome.’

The existence of at least one complete high-quality Rs sequence makes the generation of nearly complete genome sequences from other isolates a fairly inexpensive endeavor, as the existing sequence can be used as a scaffold to anchor sequences from other isolates. This not only eliminates the need to generate a scaffold (Wechter WP, Begum D, Presting G, Kim J J, Wing R A, Kluepfel D A. 2002. Physical mapping, BAC-end sequence analysis, and marker tagging of the soilborne nematicidal bacterium, Pseudomonas synxantha BG33R. OMICS 6(1):11-21) for each isolate, but also enables targeted sequencing, allowing reduction of the usual 8-10 fold sequence coverage to 1.25-2.5 fold and a significant decrease in sequencing cost. Anchoring sequences to the scaffold will be straightforward if the 91% sequence identity observed in the endoglucanase gene sequence between different isolates of Rs is representative of the level of sequence conservation across the entire genome. However, even if this is not the case, or genomes are significantly rearranged between phylotypes, most sequences can still be anchored unambiguously.

Marker Selection

With the sequences generated from phylotype III and IV strains, phylotype-specific probes are obtained for the entire genome. The sequenced Ralstonia genomes are scanned with the existing tools for oligonucleotides that a) are specific to each isolate, b) are in common between all isolates but absent in the closely related and completely sequenced genomes of Ralstonia (eutropha and metallidurans) and Pseudomonas (syringae pv tomato, syringae pv syringae, syringae pv phaseolicola, putida, aeruginosa, anaerooleophila, fluorescens). These oligonucleotides are tested for specificity using microarrays manufactured by NimbleGen Company. In addition, multiple primer pairs are selected from each isolate for PCR test development based on their melting temperature and size of hypothetical amplification product.

Oligonucleotides are selected by comparative sequence analysis of a) the published Rs sequence (race1), b) the soon-to-be-available sequences from sequevars 1 and 3 c) the complete genome sequence of three additional isolates that are sequenced and d) 600 marker sequences (spaced at 10 kb intervals throughout the genome) are amplified from ten additional isolates. These oligomers are used in the development of multiplex PCR and micro-array assays to quickly and reliably detect and differentiate isolates of Rs.

Briefly, the existing Rs sequence (GMI1000) is compared to all other completely sequenced bacterial genomes to identify 24-mers that are specific to Rs (i.e. a minimum of 3 mismatches). Sequevar1- and sequevar2-specific probes, as well as probes shared by all Rs isolates are also identified. Probes are selected based on G+C content, absence of secondary structure and even distribution throughout the genome. Up to 6,000 probes are tested on GMI1000 and isolates using the NimbleGen microarray system. A subset of these probes are selected to immediately develop PCR- or microarray-based detection methods.

A set of optimized probes is derived that will be able to differentiate between all phylotpyes, sequevars and many accessions. The bacterial wilt pathogen Ralstonia solanacearum, which is of particular interest due to its select agent status, its broad host range and geographic distribution, and murky taxonomic status. However, information learned with respect to this important pathogen is transferable to other systems and will ultimately allow the development of identification methods for all plant pathogens.

Screening of Existing Rs Collections with the Rs Micro-Array

The diagnostic Rs micro-arrays are used to screen existing Rs collections. The complete sequence of three additional, economically important Ralstonia solanacearum strains combined with the ability to screen hundreds of biologically well characterized Ralstonia solanacearum isolates will enable a global search of the genome for genes that affect geographic range and host preference.

### Example 5

Annotation of the recently sequenced Ralstonia solanacearum strain UW551 revealed the level of sequence conservation between the two R. solanacearum strains for which complete genomic sequence exists. Thus far, the analysis has yielded 221,522 potential probes that will differentiate between UW551 and GMI1000.

### Example 6

Hybridoma clones producing antibodies to plant pathogens are known in the art. Many of these have been tested for commercial application and one antibody (specific to Ralstonia solanacearum) is used in a test kit sold by Agdia, Inc. The ability to use these antibodies to enrich plant pathogens from large volumes of liquid is tested. In combination with PCR-based amplification methods this can be useful for detecting very dilute pathogen populations (e.g. in irrigation water).

### Example 7

26.77 million potential oligomers have been computationally analyzed according to the methods described herein using the genomes of Agrobacterium tumefaciens, Bradyrhizobium japonicum, Pseudomonas putida and Ralstonia solanacearum. Of 1 million randomly tested Agrobacterium probes, 2,995 (0.26%) were present in one or more other species and have been identified as potential “universal” probes. 275,893 potential probes (27.6%) have been identified as probes that could potentially react with one of the tested non-target organisms in a micro-array test. These probes can be removed from consideration to increase the specificity of the arrays.

### Example 8

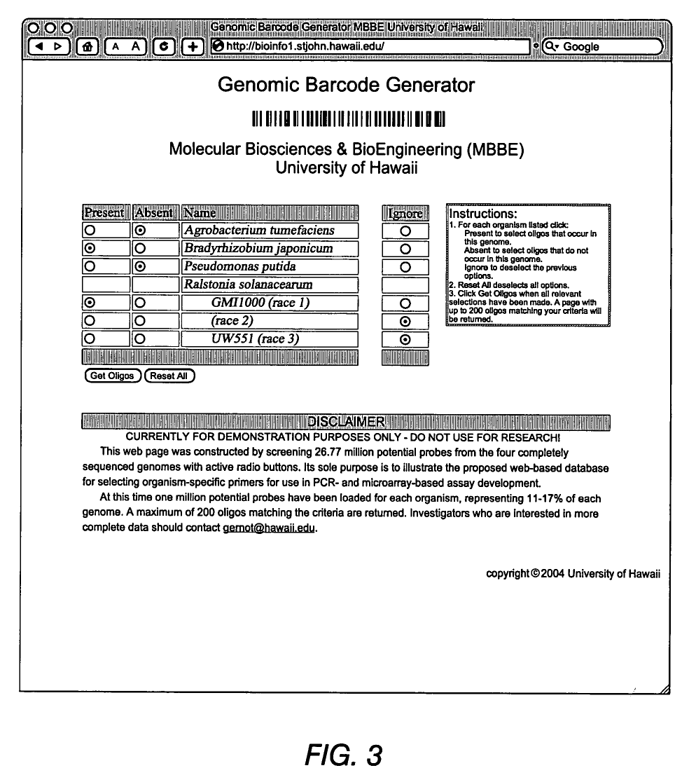

A web-based prototype “bar-coding machine” (hypertext transfer protocol: bioinfol.stjohn.hawaii.edu/) was developed that allows users to identify specific 24-mers that can be used to differentiate between selected organisms. (FIG. 3) The current version allows selection of organism-specific oligos for four soil bacteria (Agrobacterium tumefaciens, Bradyrhizobium japonicum, Pseudomonas putida and Ralstonia solanacearum), and can be expanded to include other plant pathogens, animal pathogens and human pathogens.

What is claimed is:

1. A method for identifying oligonucleotide probes for use as genetic tags in a genomic bar coding assay comprising the steps of:
selecting a nucleotide sequence in a genome of a first organism, wherein said nucleotide sequence is at least 20 nucleotides in length;
analyzing a substantially whole genome of a second organism for the presence or absence of said nucleotide sequence; and
classifying the at least one nucleotide sequence, wherein nucleotide sequences absent in the genome of the second organism are classified as taxon-specific probes and nucleotide sequences present in the genome of the second organism are classified as homologous probes.

2. The method of claim 1, wherein said nucleotide sequence is 24 nucleotides in length.

3. The method of claim 1, wherein the method further comprises the step of reverse analyzing the genome of the first organism for sequences from the genome of the second organism.

4. The method of claim 1, wherein said analyzing step comprises computational analysis.

5. The method of claim 4, wherein said analyzing step further comprises experimental analysis.

6. The method of claim 1, wherein the first and second organisms are genetically diverse members of the same species.

7. The method of claim 1, further comprising analyzing a substantially whole genome of a third organism for the presence or absence of said nucleotide sequence.

8. An array comprising a plurality of nucleic acid probes, wherein said plurality of nucleic acid probes are complementary to the oligonucleotides identified according the method of claim 1, and wherein each sequence is attached to a surface of the array in a different localized area. (PCR)

9. The array of claim 8, wherein the plurality of probes include oligonucleotides common to all organisms belonging to a sub-specific taxon but absent in closely related organisms.

10. The array of claim 8, wherein the plurality of probes comprises taxon-specific probes belonging to multiple genomic regions of a target organism.

11. The method of claim 10, wherein the multiple genomic regions are evenly distributed throughout the genome of the target organism.

12. The array of claim 8, wherein the plurality of probes comprises probes containing at least one nucleotide difference as compared to the most closely related sequence of the genome of a non-target organism.

13. The array of claim 8, wherein the plurality of probes comprises probes selected based on G+C content.

14. The array of claim 8, wherein the plurality of probes comprises probes selected based on absence of secondary structure.

15. A method definitively identifying an organism comprising:
isolating and amplifying DNA from at least one organism in a sample;
hybridizing the DNA with a set of oligonucleotide probes identified according to the method of claim 1; and
analyzing the hybridization results to determine the identity of the organism.

16. The method of claim 15, wherein said hybridizing step is performed in a single step using the array of claim 8.

17. The method of claim 15, wherein the DNA is labeled prior to the hybridization step.

18. The method of claim 15, further comprising hybridizing the DNA with the homologous probes identified according the method of claim 1 to selectively amplify taxon-specific DNA in a sample.

19. The method of claim 18, wherein said selective amplification results in increased assay sensitivity.

20. The method of claim 18, wherein the hybridizing step is performed under stringent conditions.
